(19) United States
(12) Patent Application Publication
(10) Pub. No.: US 2006/0282122 A1
(43) Pub. Date: Dec. 14, 2006

(54) TREATING CANCER WITH ELECTRIC FIELDS THAT ARE GUIDED TO DESIRED LOCATIONS WITHIN A BODY

(76) Inventor: Yoram Palti, Haifa (IL)

Correspondence Address: PROSKAUER ROSE LLP;PATENT DEPARTMENT, 1585 BROADWAY, NEW YORK, NY, 10036-8299, US

(21) Appl. No.: 11/422,998

(22) Filed: Jun. 8, 2006

Related U.S. Application Data
(60) Provisional application No. 60/688,998, filed on Jun. 8, 2005.

(51) Int. Cl. A61N 1/00

(52) U.S. Cl. 607/2

(57) ABSTRACT

Electric fields with certain characteristics have been shown to be effective at inhibiting the growth of cancer cells (and other rapidly dividing cells). However, when the cancer is located in a target region beneath the surface of a body, it can be difficult to deliver the beneficial fields to the target region. This difficulty can be surmounted by positioning a biocompatible field guide between the surface of the body and the target region, positioning electrodes on either side of the field guide, and applying an AC voltage with an appropriate frequency and amplitude between the electrodes. This arrangement causes the field guide to route the beneficial field to the target region. In an alternative embodiment, one of the electrodes is positioned directly on top of the field guide.

### CROSS REFERENCE TO RELATED APPLICATIONS

This application claims priority to U.S. provisional application No. 60/688,998, filed Jun. 8, 2005.

### BACKGROUND

U.S. Pat. No. 6,868,289, which is incorporated herein by reference, discloses methods and apparatuses for treating tumors using an electric field with particular characteristics. It also discloses various ways to modifying the electric field intensity at desired locations (see, e.g., FIGS. 21-26).

This application discloses additional ways for modifying the field so as to significantly increase or decrease it at desired locations in a patient's body. These modifications can improve the quality and selectivity of treatment of lesions and tumors and improve selective tissue ablation or destruction.

FIG. 1A shows an arrangement where two electrodes 11, 11′ are placed on the patient's skin 15 above the underlying tissue 10 (e.g., muscle) in an environment of air 16. FIG. 1B depicts the results of a finite element simulation of the electric field generated in the air and in the muscle tissue, when the insulated electrodes 11, 11′ are positioned on the skin 15 as shown in FIG. 1A, and a 100 kHz AC signal is applied to the electrodes. Preferably, the insulated electrodes have a conductive core and an insulating layer with a high dielectric constant as described in U.S. Pat. No. 6,868,289, and they are configured to contact the surface of the body with the insulating layer disposed between the conductive core and the surface of the body.

FIG. 1B, (like all the other field intensity maps included herein) shows the field intensity in mV/cm when 1 Volt AC (measured zero-to-peak) is induced between the proximal side of the tissue just beneath the first electrode and the proximal side of the tissue just beneath the second electrode (by applying a sufficiently large voltage between the electrodes' terminals). The numbers along the x and y axes in the main section of FIG. 1B (and in the other field intensity maps included herein) represent distance measured in cm. Each contour line represents a constant step size down from the 1 V peak, and the units are given in mV/cm. Note, however, that because the voltage changes so rapidly at the higher values, the contour lines run together to form what appears to be a solid black region.

It is seen in FIG. 1B that, both in the air above the skin 15 and the tissue below the skin 15, the field intensity is maximal in regions that are close to the edges of the electrodes 11, 11′ and that the field intensity is attenuated rapidly with distance. As a result, if a tumor lies relatively deep below the skin 15, it may be difficult to deliver the desired field strength that is needed for effective treatment to that tumor to the target region.

A similar situation exists in the human head. FIG. 2 is a schematic representation of a human head 5 in which all tissue components are given their corresponding electric properties. The head includes skin 1, bone 2, gray matter 3 and white matter 4. FIG. 3A is a schematic representation of the positioning of the electrodes 11, 11′ on the skin surface on the same side of the head, and FIG. 3A shows the electric field that is generated under those conditions when a 100 kHz AC field is applied between the electrodes. (The field calculation was done by a finite element simulation based on the schematic representation of the head shown in FIG. 2.) The field intensity is highest in the vicinity of the electrodes in the skin and the superficial areas of the brain and drops rapidly. Notably, the field strength near the middle of the head is very weak (i.e., less than 20 mV/cm).

FIG. 4A is a schematic representation of the positioning of the electrodes 11, 11′ on opposite sides of a human head, and FIG. 4B shows the electric field that is generated under those conditions when a 100 kHz AC field is applied between the electrodes. Once again, the field calculation was done by a finite element simulation, and once again, the field strength near the middle of the head is very weak (i.e., less than 24 mV/cm). The field intensity is highest in the vicinity of the electrodes in the skin and the superficial areas of the brain and drops rapidly, so that the field intensity is relatively low at the center of the head. Thus, the treatment efficacy of the field for any tumor or lesion at a distance from the surface or electrodes would be correspondingly diminished.

### SUMMARY

A biocompatible field guide is positioned between the surface of the body and the target region beneath the surface. Electrodes are positioned on either side of the field guide, and an AC voltage with an appropriate frequency and amplitude is applied between the electrodes so that the field guide routes the electric field to the target region. In an alternative embodiment, one of the electrodes is positioned directly on top of the field guide.

### BRIEF DESCRIPTION OF THE DRAWINGS

FIG. 1A is a schematic representation of two electrodes placed on a patient's skin above a target region.

FIG. 1B shows the electric field that results from the FIG. 1A arrangement.

FIG. 2 is a schematic representation of a human head.

FIG. 3A is a schematic representation two electrodes positioned on the same side of the head.

FIG. 3B shows the electric field that results from the FIG. 3A arrangement.

FIG. 4A is a schematic representation two electrodes positioned on opposite sides of the head.

FIG. 4B shows the electric field that results from the FIG. 4A arrangement.

FIGS. 5A and 5B are section and plan views, respectively, of a first embodiment of the invention using a solid insulated rod.

FIG. 6A shows the electric field that results from the FIG. 5 arrangement.

FIG. 6B is a magnified view of the center of FIG. 6A.

FIG. 7A shows the electric field for a second embodiment using a hollow insulated rod.

FIG. 7B is a magnified view of the center of FIG. 7A.

FIG. 8A shows the electric field for the third embodiment when a conductive gel is added.

FIG. 8B is a magnified view of the center of FIG. 8A.

FIG. 9A shows the electric field for a third embodiment using a hollow conducting rod.

FIG. 9B is a magnified view of the center of FIG. 9A.

FIG. 9C depicts a set of field strength plots for six hollow metal tube field guides.

FIG. 10A shows the electric field that results from using a solid conducting rod.

FIG. 10B is a magnified view of the center of FIG. 10A.

FIGS. 11A and 11B are section and plan views, respectively, of a fourth embodiment of the invention using a solid insulated bead.

FIG. 12A shows the electric field that results from the FIG. 11 arrangement.

FIG. 12B is a magnified view of the center of FIG. 12A.

FIG. 13A shows the electric field for a fifth embodiment using a hollow conducting bead.

FIG. 13B is a magnified view of the center of FIG. 13A.

FIG. 14 shows the electric field for a sixth embodiment in which a conductive gel is placed on the surface of the skin between the electrodes.

FIG. 15 shows the electric field for an alternative arrangement in which a rod-shaped field guide is placed directly beneath one of the electrodes.

FIG. 16 shows a curved field guide that guides the field to a target area without passing through a vital organ.

### DESCRIPTION OF THE PREFERRED EMBODIMENTS

The inventor has recognized that the field can be guided to the desired location in the patient's body using appropriate field guides.

In some embodiments of the invention, an insulating member is introduced into the medium or tissue in a position that enables the member to act as a Field Guide (FG) in the given medium. While elongated shapes such as rods, tubes, bars, or threads are preferred, other shapes (e.g., sheets or plates) may also be used. In these embodiments, the electric impedance of the FG, Z_{FG} is significantly higher than that of the medium Z_{FG} (Z_{FG}>>Z_{M}). For example, the FG may be made of a dielectric insulating material such as plastic (e.g. polystyrene, PVC, Teflon), silicone, rubber, etc., while the medium is tissue (e.g., muscle). Insulators with a very high dielectric constant (see the electrode insulations of the '289 patent) may be preferable to those with low dielectric properties. For use in medical application, the FG should preferably be made of a biocompatible material. Optionally, the FG may be made of a biodegradable material, as long as the electrical properties remain as described herein.

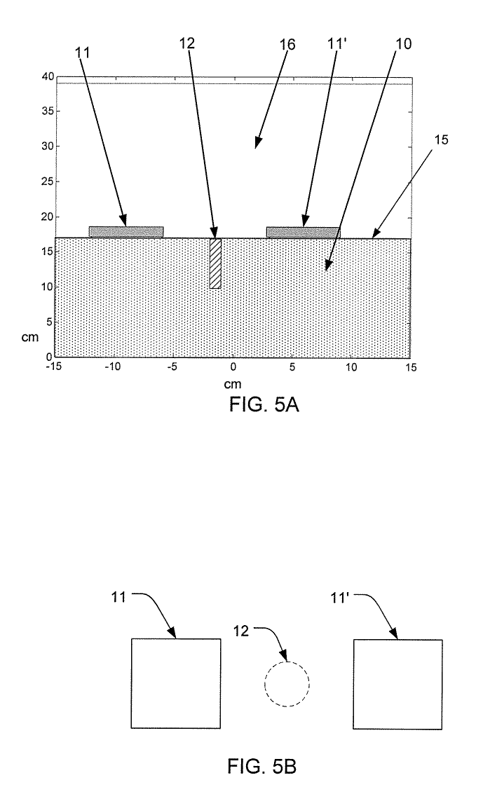

FIGS. 5A and 5B are section and plan views of a first embodiment in which an insulated rod 12 is inserted into tissue 10 between a pair of insulated electrodes 11, 11′. The upper end of the FG rod 12 is positioned just under the skin 15. The preferred diameter for the rod is between about 0.5 mm and about 10 mm, but diameters outside of that range may also be used.

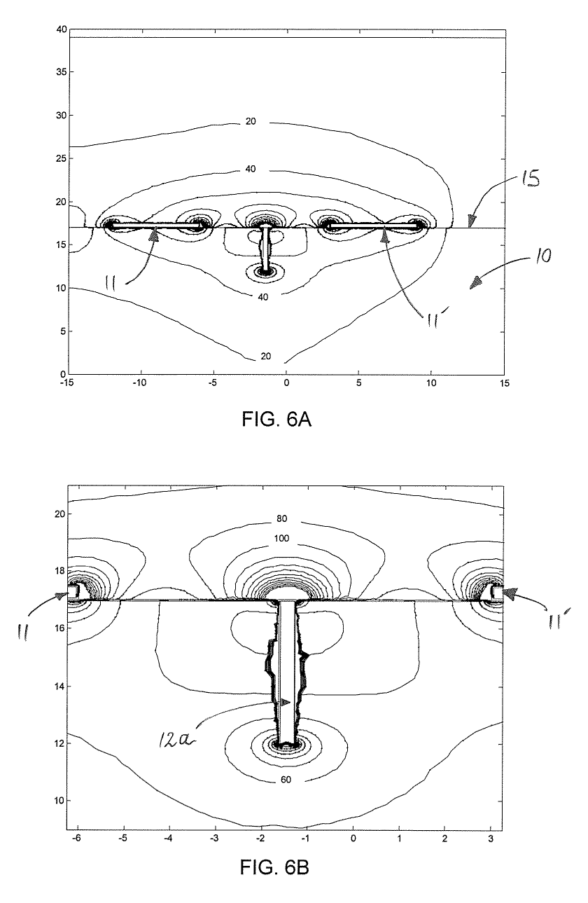

FIG. 6A shows a finite element simulation of the electric field that is generated in the tissue when a 5 cm long, 3 mm diameter, insulated FG rod 12a made of solid plastic with an impedance between 4-6 orders of magnitude higher than the impedance of the tissue and a dielectric constant of about 2-3 is inserted into the tissue 10 between the pair of insulated electrodes 11, 11′. The upper (proximal) ends of the electrodes are located on the skin surface, and a 100 kHz AC voltage is applied between the electrodes. FIG. 6B is a magnified portion of the center of FIG. 6A, to show the field in greater detail. As seen in FIGS. 6A and 6B, the strength of the field is much higher just below the rod 12a. Thus, by inserting the FG so that it sits right above the desired target location, the field is directed to the desired location, along with the corresponding beneficial effects of that field (as described in the '289 patent).

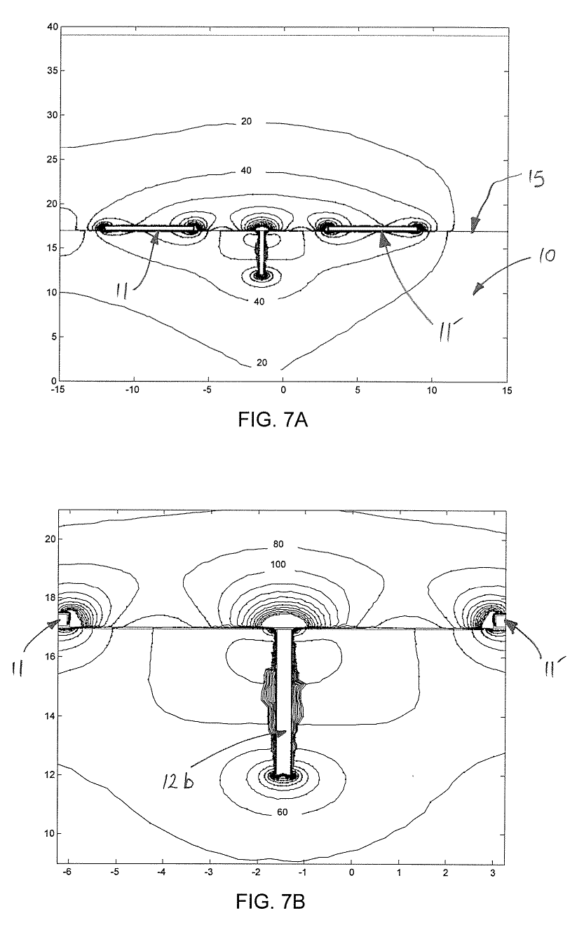

The second embodiment is similar to the first embodiment, except that a hollow insulated rod 12b is used in place of the solid insulated rod 12a of the first embodiment. The rod in this example has an outer diameter of 3 mm and an inner diameter of 2.5 mm, and is also 5 cm long. FIG. 7A shows a finite element simulation of the electric field for this second embodiment, and FIG. 7B shows a magnified view of the center of FIG. 7A. Here again, the strength of the field is much higher just below the rod. We therefore see that a hollow insulating FG can also be used to direct the field to a desired location.

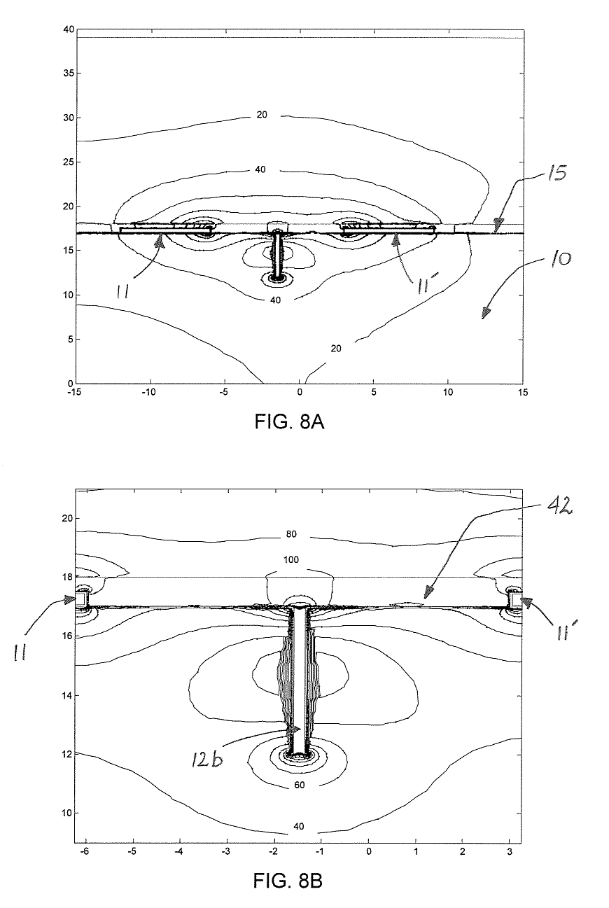

Optionally, conductive gel may be placed on the surface of the skin in the region between the insulated electrodes. FIG. 8A shows a finite element simulation of the electric field for the second embodiment (using the hollow insulated rod 12b) when conductive gel 42 is spread on the skin between the electrodes 11, 11′, and FIG. 8B shows a magnified view of the center of FIG. 8A. Here again, the strength of the field is much higher just below the rod. In addition, the field is also stronger in the region between the electrodes just below the surface of the skin 15 beneath the gel 42. Note that the conductive gel described in connection with this embodiment may also be used in the other embodiments described herein, with similar results.

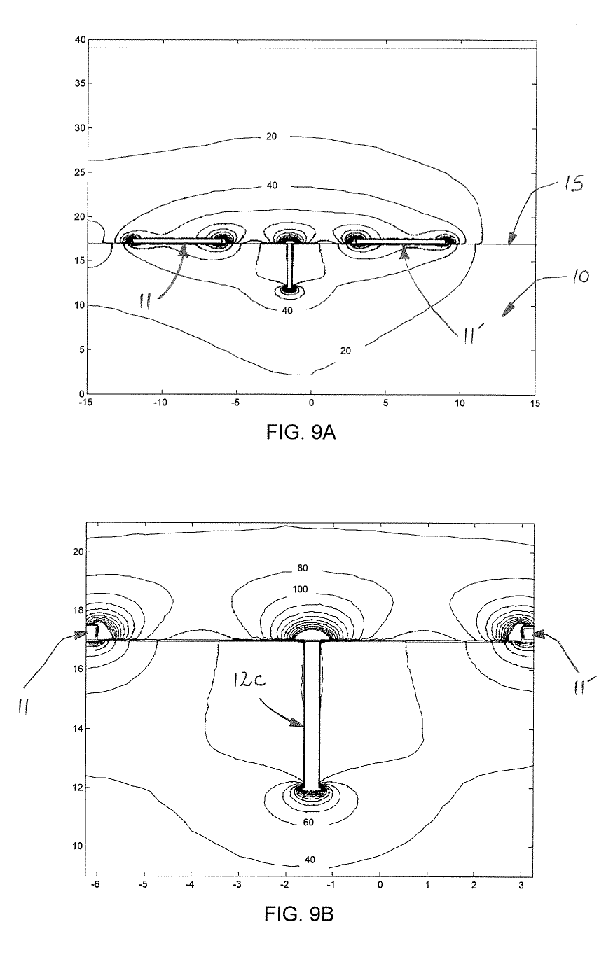

In a third embodiment, a hollow conducting rod is used instead of the hollow insulating rod of the second embodiment. In this third embodiment, the electric impedance of the FG, Z_{FG} is significantly lower than that of the medium Z_{M} (Z_{FG}<<Z_{M}). For example, FG may be made of metal such as gold, stainless steel, titanium, etc., while the medium is tissue (e.g., muscle). FIGS. 9A shows a finite element simulation of the electric field for this third embodiment using a hollow conducting rod 12c, and FIG. 9B shows a magnified view of the center of FIG. 9B. Here again, the strength of the field is much higher just below the rod 12c. We therefore see that a hollow conducting FG can also be used to direct the field to a desired location.

FIG. 9C depicts a set of field strength plots for six hollow metal tube FGs with six different diameters (each having a length of 5 cm) plus a seventh, flat, field strength plot for the case when no FG is used. Each plot depicts how the field strength at the depth of the tube varies as a function of horizontal distance from the center of the tip of the tube. As seen in FIG. 9C, the widest plot corresponds to the tube with the 5 mm inner diameter, and successively narrower plots correspond to tubes with inner diameters of 4, 3, 2, 1, and 0.5 mm, respectively. As between the depicted plots, the maximum field strength at the center of the tip of the FG occurs for the 2 mm diameter tube.

In alternative embodiments (not shown), the FG can be of compound construction, such as a hollow metal rod that is coated with insulation or a layer of biocompatible material. In other alternative embodiments, instead of sinking the rod into the tissue to a depth where the top of the rod is just beneath the surface of the patient's skin, a rod that protrudes through the skin may be used with a similar level of effectiveness. In those embodiments, it is advisable to take suitable precautions to reduce the risk of infection.

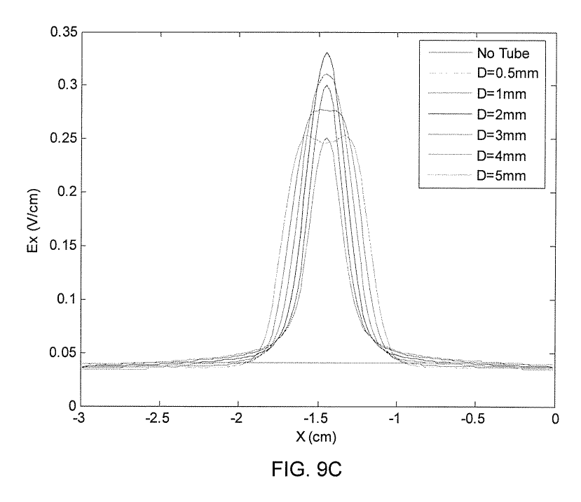

In the above-describe embodiments, the FGs are seen to be effective in carrying the field into deep parts of the tissue. In contrast, if a solid conducting rod 12d were to be used, the field would not be directed to below the bottom of the rod, as shown in the finite element simulation of FIGS. 10A and 10B.

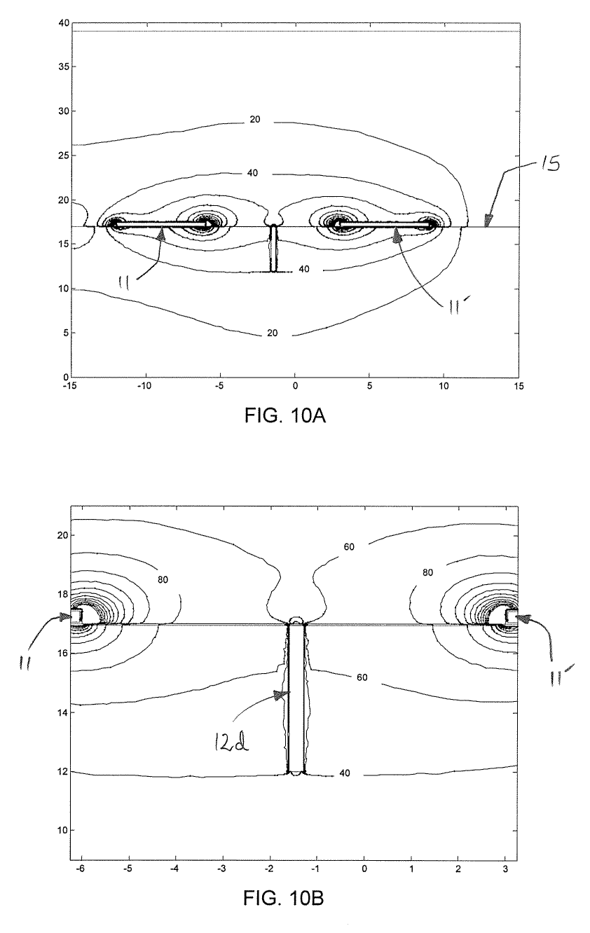

FIGS. 11A and 11B are section and plan views of a fourth embodiment of the invention. In this embodiment, a short insulated solid FG bead 22 is inserted just below the skin 15 between two insulated electrodes 11, 11′. The same materials that are suitable for the insulated FG rod 12a described above in connection with the first embodiment are also suitable for this insulated bead 22. The bead in the illustrated example of this embodiment is cylindrical with a 1 cm length and an outer diameter of 1 cm. Other shapes for the bead (e.g., a cube) may be used as well.

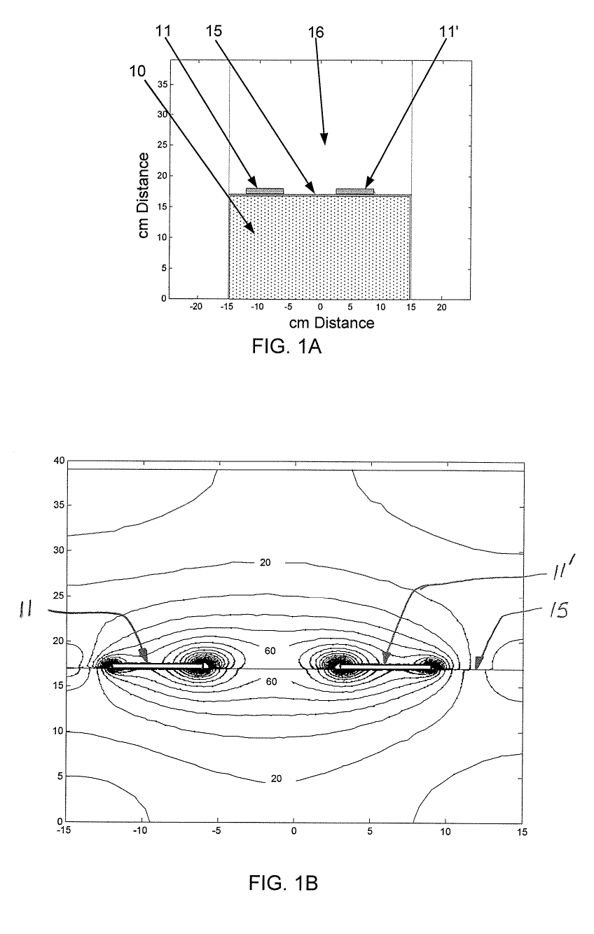

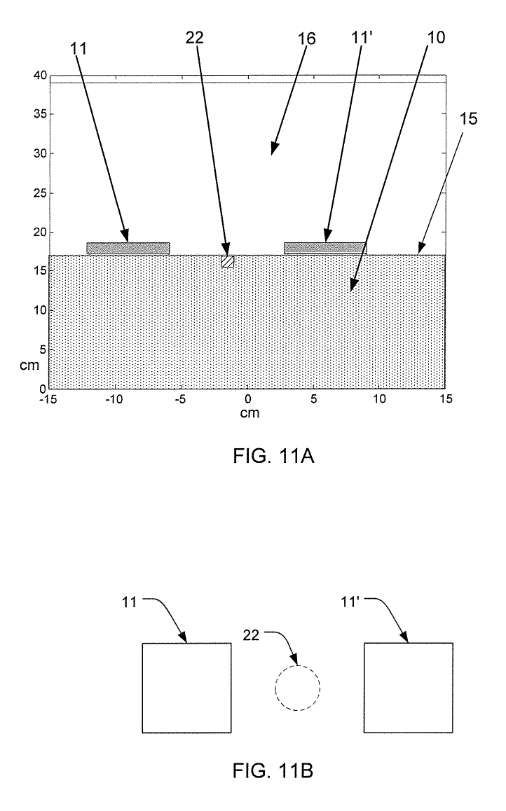

FIG. 12A shows a finite element simulation of the electric field that is generated in the tissue when the insulated bead 22 is inserted beneath the skin into the tissue 10 between the pair of insulated electrodes 11, 11′. The electrodes are located on the skin surface, and a 100 kHz AC voltage is applied between the electrodes. FIG. 12B is a magnified portion of the center of FIG. 12A, to show the field in greater detail. As seen in FIGS. 12A and 12B, the strength of the field is higher beneath the surface of the skin as compared to when there is no FG, as shown in FIGS. 1A and 1B. This embodiment is therefore useful for directing the field into shallow tumors such as malignant melanoma skin lesions or skin metastases from breast cancer, etc.

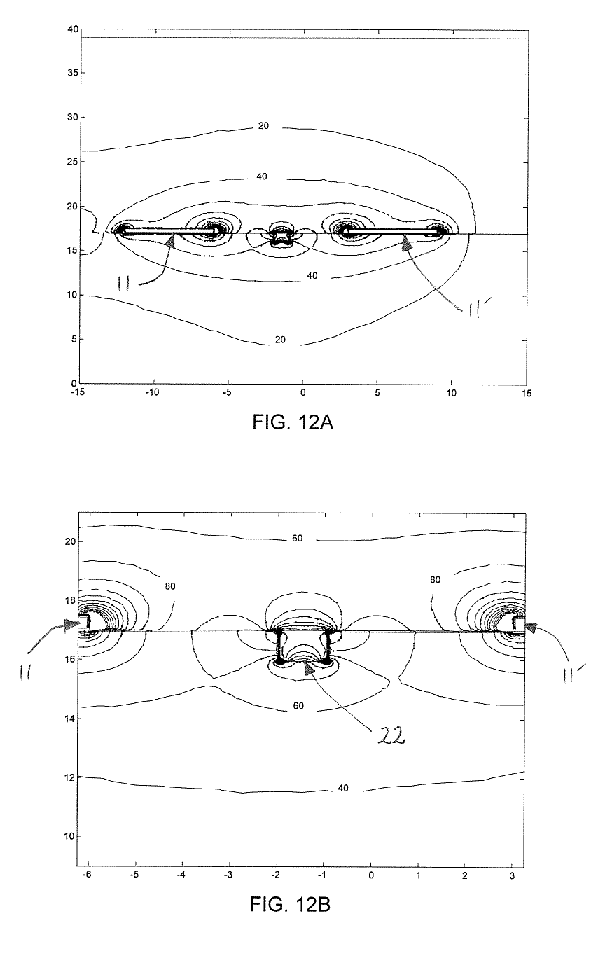

FIG. 13A and 13B show the normal and magnified views of a finite element simulation of the electric field that is generated in the tissue in a fifth embodiment in which the insulated bead 22 of the previous embodiment is replaced with a hollow conductive bead 32. The field strength in this embodiment is also higher beneath the surface of the skin as compared to when there is no FG, as shown in FIGS. 1A and 1B. This fifth embodiment is therefore also useful for directing the field into shallow tumors.

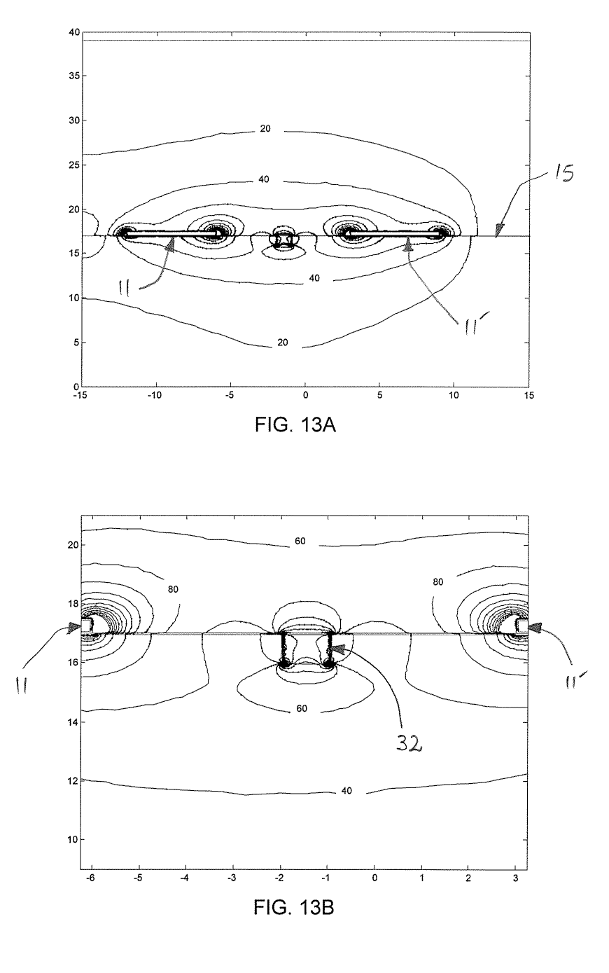

FIG. 14 illustrates a sixth embodiment, in which a conductive FG is placed on the skin between the insulated electrodes 11, 11′, in parallel with the skin surface. In the illustrated embodiment, the conductive FG is a conductive gel 42 that is spread on the skin in a continuous layer in the region beneath and between the electrodes. Preferably, the gel has high conductivity and is biocompatible for extended periods of time. One suitable gel is AG603 Hydrogel, which is available from AmGel Technologies, 1667 S. Mission Road, Fallbrook, Calif. 92028-4115, USA. In comparison to the case with no conductive gel (as seen in FIG. 1B), there is a marked intensification of the field in the skin 15 and subcutaneous tissues 10 in the region between the two electrodes 11, 11′.

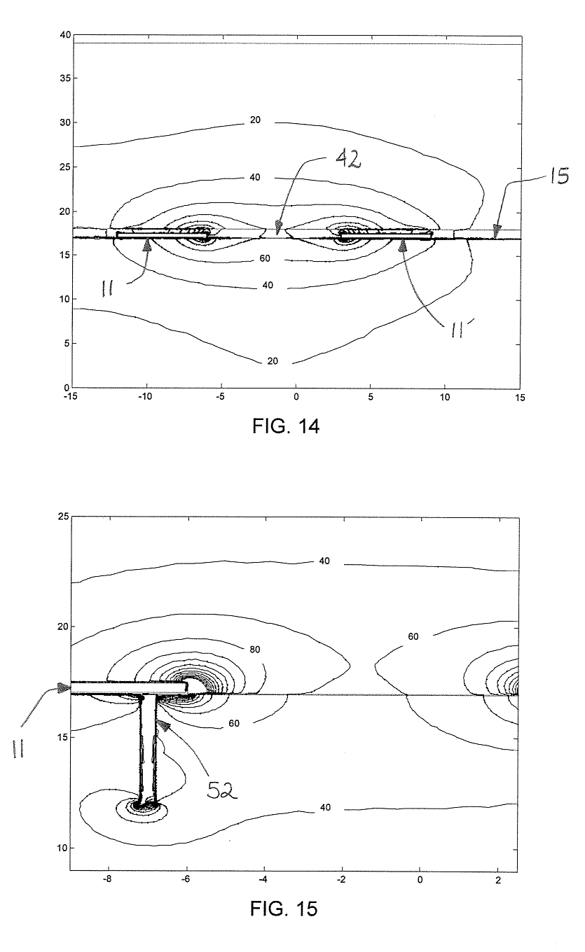

In a variation of the above-describe embodiments, instead of placing the FG between the electrodes as it is in FIGS. 5-9, and FG rod or bead 52 (which may be either solid insulating, hollow insulating, or hollow conductive, as described above) is placed directly beneath one of the insulated electrodes 11. FIG. 15 shows a finite element simulation of the electric field for this configuration. Once again, the strength of the field is much higher just beneath the FG than it is at a corresponding depth when no FG is used, as shown in FIG. 1B.

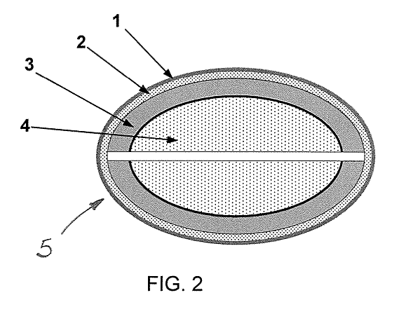

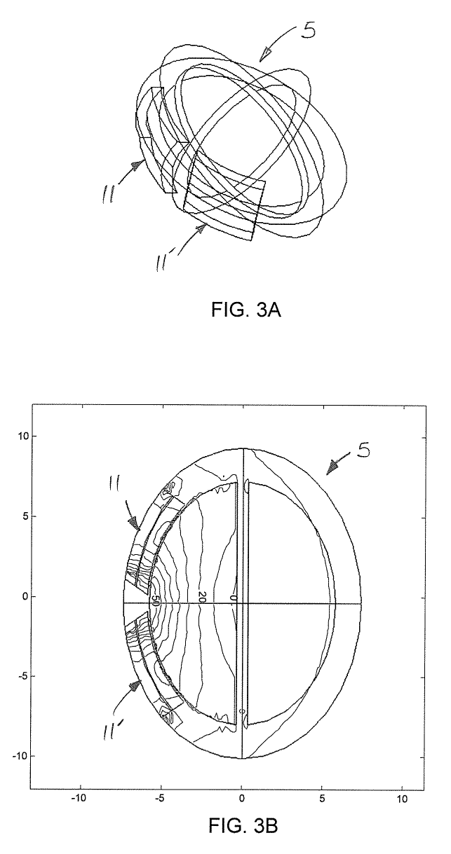

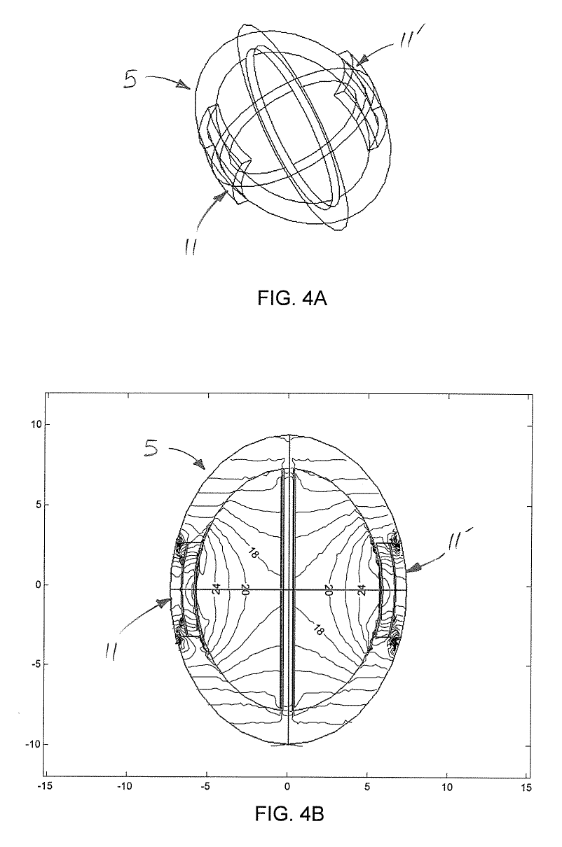

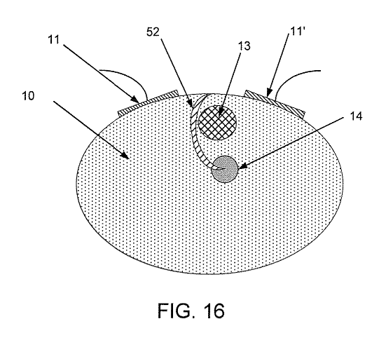

Although straight FGs are depicted in FIGS. 1-10, other shapes may be used in alternative implementations, as appropriate for the anatomy in the vicinity of the tumor. In FIG. 16, for example, a curved FG 52 is used to circumnavigate a vital organ 13 (to avoid piercing the organ 13 with a straight FG) on its way to a target area 14. A thin flexible FG that resembles monofilament fishing line may also be used, in which case it can be threaded into the desired location using a guiding device that is appropriate for the anatomical region.

Superficial FGs may be positioned on the skin surface, under the surface, passing through the skin, or a combination thereof. The superficial conducting FG can be a gel sheet, metal sheet, rod tube, etc. The FG can be inserted and maneuvered into position by means of a hypodermic needle, a guided catheter-like device, an incision, etc. Optionally, a combination of active electrodes, superficial FGs, and internal FGs may be used as required to obtain the desired field.

Although the above-described embodiments are explained in the context of increasing the field strength at certain locations in the tissue, a side effect of the FGs is that the field strength is decreased in other areas. This situation can be exploited by using FGs to create areas with lower field intensities so as to avoid effecting, stimulating, or heating sensitive areas within the body or tissue. This provides the ability to protect a sensitive region without depending on the shielding effects of closed or partially closed conductors surrounding an element (such as the conductive net that surrounds a sensitive organ, as described in the '289 patent). Examples of the creation of a reduced-field region in the form of a ring (30) or doughnut can be envisioned by extending the cross sections of FIGS. 6, 7, and 9 out to three dimensions, in which case it becomes clear that a low field area surrounds the FG (as compared to the higher field intensities when there is no FG, as shown in FIG. 1B).

The described use of FGs can increase the efficacy of treating tumors or lesions in many deeply located body locations including, for example, the brain, lung, colon, liver, pancreas, breast, prostate, ovaries, etc. The optimum frequency and field strength will vary depending on the particular problem being treated. For many types of cancers, frequencies between 100 kHz and 300 kHz at field strengths between 1 and 10 V/cm have been shown to be helpful. Examples include B16F1 melanoma, which is susceptible to 120 kHz fields; and F-98 glioma, which is susceptible to fields between 150 and 250 kHz. See E. D. Kirson et al., Disruption of Cancer Cell Replication by Alternating Electric Fields, Cancer Research 64, 3288-3295, May 1, 2004, which is incorporated herein by reference.

I claim:

1. An apparatus for inhibiting growth of rapidly dividing cells located in a target region beneath the surface of a body, the apparatus comprising:
a biocompatible field guide having (a) a proximal end, (b) a distal end, and (c) an impedance that is either much higher or much lower than the impedance of the body, wherein the distal end is positioned adjacent to the target region and the proximal end is positioned near or above the surface of the body;
a first electrode positioned on the surface of the body on a first side of the field guide;
a second electrode positioned on the surface of the body on a second side of the field guide; and
an AC voltage source configured to generate an AC voltage between the first electrode and the second electrode, wherein the frequency and amplitude of the AC voltage and the impedance of the field guide have values that result in the formation of an electric field in the target region that inhibits the growth of the rapidly dividing cells.

2. The apparatus of claim 1, wherein the field guide is rod-shaped.

3. The apparatus of claim 1, wherein the field guide is curved.

4. The apparatus of claim 1, wherein the first and second electrodes each have a conductive core and an insulating layer with a high dielectric constant, and wherein the first and second electrodes are adapted to contact the surface of the body with the insulating layer disposed between the conductive core and the surface of the body.

5. The apparatus of claim 1, wherein the AC voltage has a frequency between 100 kHz and 300 kHz.

6. The apparatus of claim 5, wherein the electric field in the target region has a field strength greater than 1 V/cm.

7. The apparatus of claim 1, wherein the impedance of the field guide is much higher than the impedance of the body.

8. The apparatus of claim 7, wherein the first and second electrodes each have a conductive core and an insulating layer with a high dielectric constant, and wherein the first and second electrodes are adapted to contact the surface of the body with the insulating layer disposed between the conductive core and the surface of the body.

9. The apparatus of claim 7, wherein the AC voltage has a frequency between 100 kHz and 300 kHz.

10. The apparatus of claim 9, wherein the electric field in the target region has a field strength greater than 1 V/cm.

11. A method of inhibiting growth of rapidly dividing cells located in a target region beneath the surface of a body, the method comprising:
positioning a biocompatible field guide, the field guide having (a) a proximal end, (b) a distal end, and (c) an impedance that is either much higher or much lower than the impedance of the body, so that the distal end is adjacent to the target region and the proximal end is near or above the surface of the body;
positioning a first electrode on the surface of the body on a first side of the field guide;
positioning a second electrode on the surface of the body on a second side of the field guide; and
applying an AC voltage between the first electrode and the second electrode, wherein the frequency and amplitude of the AC voltage and the impedance of the field guide have values that result in the formation of an electric field in the target region that inhibits the growth of the rapidly dividing cells.

12. The method of claim 11, wherein the impedance of the field guide is much higher than the impedance of the body.

13. The method of claim 12, wherein the first and second electrodes each have a conductive core and an insulating layer with a high dielectric constant, and wherein the first and second electrodes are adapted to contact the surface of the body with the insulating layer disposed between the conductive core and the surface of the body.

14. The method of claim 12, wherein the AC voltage has a frequency between 100 kHz and 300 kHz.

15. The method of claim 14, wherein the electric field in the target region has a field strength greater than 1 V/cm.

16. An apparatus for inhibiting growth of rapidly dividing cells located in a target region beneath the surface of a body, the apparatus comprising:
a biocompatible field guide having (a) a proximal end, (b) a distal end, and (c) an impedance that is either much higher or much lower than the impedance of the body, wherein the distal end is positioned adjacent to the target region and the proximal end is positioned near or above the surface of the body;
a first electrode positioned on the surface of the body directly above the field guide;
a second electrode positioned on the surface of the body off to a side of the field guide; and
an AC voltage source configured to generate an AC voltage between the first electrode and the second electrode, wherein the frequency and amplitude of the AC voltage and the impedance of the field guide have values that result in the formation of an electric field in the target region that inhibits the growth of the rapidly dividing cells.

17. The apparatus of claim 16, wherein the impedance of the field guide is much higher than the impedance of the body.

18. The apparatus of claim 17, wherein the first and second electrodes each have a conductive core and an insulating layer with a high dielectric constant, and wherein the first and second electrodes are adapted to contact the surface of the body with the insulating layer disposed between the conductive core and the surface of the body.

19. The apparatus of claim 17, wherein the AC voltage has a frequency between 100 kHz and 300 kHz.

20. The apparatus of claim 19, wherein the electric field in the target region has a field strength greater than 1 V/cm.

21. A method of inhibiting growth of rapidly dividing cells located in a target region beneath the surface of a body, the method comprising:
positioning a biocompatible field guide, the field guide having (a) a proximal end, (b) a distal end, and (c) an impedance that is either much higher or much lower than the impedance of the body, so that the distal end is adjacent to the target region and the proximal end is near or above the surface of the body;
positioning a first electrode on the surface of the body directly above the field guide;
positioning a second electrode on the surface of the body off to a side of the field guide; and
applying an AC voltage between the first electrode and the second electrode, wherein the frequency and amplitude of the AC voltage and the impedance of the field guide have values that result in the formation of an electric field in the target region that inhibits the growth of the rapidly dividing cells.

22. The method of claim 21, wherein the impedance of the field guide is much higher than the impedance of the body.

23. The method of claim 22, wherein the first and second electrodes each have a conductive core and an insulating layer with a high dielectric constant, and wherein the first and second electrodes are adapted to contact the surface of the body with the insulating layer disposed between the conductive core and the surface of the body.

24. The method of claim 22, wherein the AC voltage has a frequency between 100 kHz and 300 kHz.

25. The method of claim 24, wherein the electric field in the target region has a field strength greater than 1 V/cm.
